(19) United States
(12) Patent Application Publication
(10) Pub. No.: US 2006/0169294 A1
(43) Pub. Date: Aug. 3, 2006

(54) Inertial navigation method and apparatus for wireless bolus transit monitoring in gastrointestinal tract

(76) Inventors: Karan V.I.S. Kaler, N.W. Calgary (CA); Martin P. Mintchev, Calgary (CA)

Correspondence Address: LAW OFFICE OF MARC D. MACHTINGER, LTD., 750 W. LAKE COOK ROAD, SUITE 350, BUFFALO GROVE, IL, 60089, US

(21) Appl. No.: 11/304,365

(22) Filed: Dec. 15, 2005

Related U.S. Application Data
(60) Provisional application No. 60/636,474, filed on Dec. 15, 2004.

(51) Int. Cl. A61B 19/00; A61B 5/117; A61B 5/103

(52) U.S. Cl. 128/899; 600/593; 128/903

(57) ABSTRACT

A swallowable electronic bolus or “pill” that can monitor any one or more of the following parameters in real time as the bolus moves through the esophagus: (i) acceleration of the bolus; (ii) velocity of the bolus; (iii) position of the bolus; and (iv) contractile force on the bolus that pushes it through the gastrointestinal tract.

### CROSS-REFERENCE TO RELATED APPLICATIONS

This application claims the benefit under 35 USC 119(e) of provisional application No. 60/636,474 filed Dec. 15, 2004.

### BACKGROUND

1. Field of the Invention

The present invention is related to methods and apparatus for monitoring bolus movement and diagnosing maladies in gastrointestinal tracts, and more specifically to tetherless apparatus and methods for monitoring acceleration, velocity of propagation, position, and peristaltic force on boluses being moved through gastrointestinal tracts.

2. Background of the Invention

Monitoring and collecting data on bolus transit in gastrointestinal tracts, both antegrade (proximal to distal) and retrograde (distal or proximal), is useful in diagnosing maladies and dysfunctions in the gastrointestinal tract, such as in the pharynx, esophagus, small intestine, and/or large intestine. For example, monitoring bolus transit in the esophagus has been an essential tool for diagnosis of achalasia, diffuse esophageal spasm, abnormalities associated with systemic esophageal disorders, and gastroesophageal reflux disease (GERD). In adults, the esophagus is an approximately 25 cm long muscular tube that can be divided into three functional regions, the upper esophageal sphincter (UES), the mid-section or body of the esophagus, and the lower esophageal sphincter (LES). The pharynx extends from the oral cavity to the larynx, where it becomes continuous with the esophagus, and the LES isolates the stomach from the mid-section of the esophagus. When a person swallows a quantity of solid or liquid matter, often called a bolus, the bolus is propelled by the tongue from the oral cavity (mouth) and through the pharynx to the esophagus. The UES is supposed to open to allow the bolus into the body of the esophagus and then close again behind the bolus to propel the bolus and to isolate the esophagus from the pharynx. Peristaltic contractions of the muscular tube that forms the mid-section of the esophagus are also supposed to propel the bolus through the mid-section of the esophagus toward the stomach. The LES is supposed to open when the peristaltic contractions approach the LES to allow the bolus to enter the stomach, whereupon the LES is supposed to close again to isolate the esophagus from the stomach.

The actual physiological behavior of the UES, mid-section, and LES regions of the esophagus can be assessed by manometry with various degrees of technique-related variability. Bolus transit can be monitored also using barium radiography and the recently suggested multichannel intraluminal impedancometry. Barium radiography has been a routine diagnostic tool for monitoring esophageal bolus transit. Although the patient and staff exposure to the potentially harmful effects of X-Ray radiation has been significantly reduced with the recent advances in X-Ray imaging technology, this technique is not completely safe and non-invasive for both the patient and the medical staff performing the testing.

Recently, impedance-based methods for intraluminal esophageal studies have been intensively investigated and commercialized. Although this development represents a significant step forward in monitoring esophageal physiology in health and disease, it is still invasive, which remains a source of constant discomfort for both patients and medical professionals alike, not to mention the associated increased costs. It is also interesting to note the tendency of increasing the number of channels in intraluminal monitoring, which is a clear indicator that in an ideal case scenario, continuous picture of pressure distribution and dynamics throughout the entire esophagus would be an important diagnostic asset.

In 1997, Iddan et al. proposed an in vivo video camera system that uses a swallowable electronic capsule for imaging areas of interest in the entire digestive tract. The endoscopy capsule comprises of (1) a camera system; (2) an optical system for imaging an area of interest onto the camera; and (3) a transmitter which transmits the video output. This in vivo video camera system operates as an autonomous video endoscope, and internal images of the digestive tract can be delivered wirelessly to a reception system. However, such a system lacks in providing kinematic information of the capsule inside the human body.

In 2002, Spelman et al. proposed an apparatus and method for monitoring gut motility using an ingestible magnet. With this non-invasive method, the position of the magnet in the gut can be determined using a compass external to the patient. However, more comprehensive information including velocity, acceleration, and peristaltic force dynamics was not measured.

### SUMMARY OF THE INVENTION

An object of this invention, therefore, is to provide a swallowable electronic bolus or “pill” that can monitor any one or more of the following parameters in real time as the bolus moves through the esophagus: (i) acceleration of the bolus; (ii) velocity of the bolus; (iii) position of the bolus; and (iv) contractile force on the bolus that pushes it through the gastrointestinal tract. Thus according to an aspect of the invention, there is provided an apparatus for monitoring transit of a bolus in a gastrointestinal tract of a test subject's body, comprising a capsule sized and shaped for being swallowable and movable through the gastrointestinal tract, said capsule containing inertial navigation sensors (e.g., accelerometers and gyroscopes) that are capable of sensing and measuring acceleration, velocity and position of the capsule on a real time basis as the capsule moves through the gastrointestinal tract or through portions of the gastrointestinal tract. According to a further aspect of the invention, there is provided a method of monitoring bolus movement through at least a portion of a gastrointestinal tract of a test subject, comprising the test subject swallowing a set of inertial navigation sensors, which measures all or at least one of the following parameters: acceleration, velocity and position on a real time basis, produces electric signals that are indicative of such measurements; and collects the acceleration measurements for analysis and display.

Another object of the invention is to provide such an electronic bolus that can communicate data indicative of such parameters from inside the gastrointestinal tract to a receiver positioned outside of a subject's body.

These and other objects are described herein or will be apparent to persons skilled in the art upon reading the description and statements of the invention.

### BRIEF DESCRIPTION OF THE DRAWINGS

The accompanying drawings, which are incorporated in and form a part of the specification, illustrate the preferred embodiments of the present invention, involving a single inertial navigation sensor, an accelerometer, and together with the descriptions serve to explain the principles of the invention.

In the drawings:

FIG. 1 is a diagrammatic view of an electronic capsule bolus being swallowed by a person and communicating acceleration data to a receiver outside the person's body for recording in a data logger according to this invention;

FIG. 2 is an enlarged isometric view of the capsule shown with a portion of the housing wall cut away to reveal the components inside;

FIG. 3 is a diagrammatic view similar to FIG. 1, but showing the electronic capsule bolus in the UES region of the esophagus; and

FIG. 4 is a diagrammatic view similar to FIGS. 1 and 2, but showing the electronic capsule bolus in the mid-section of the esophagus.

FIG. 5 shows the anticipated plot that describes the bolus acceleration in a swallowing process.

FIG. 6 shows an exemplary inertial navigation sensor.

### DETAILED DESCRIPTION OF THE PREFERRED EMBODIMENTS

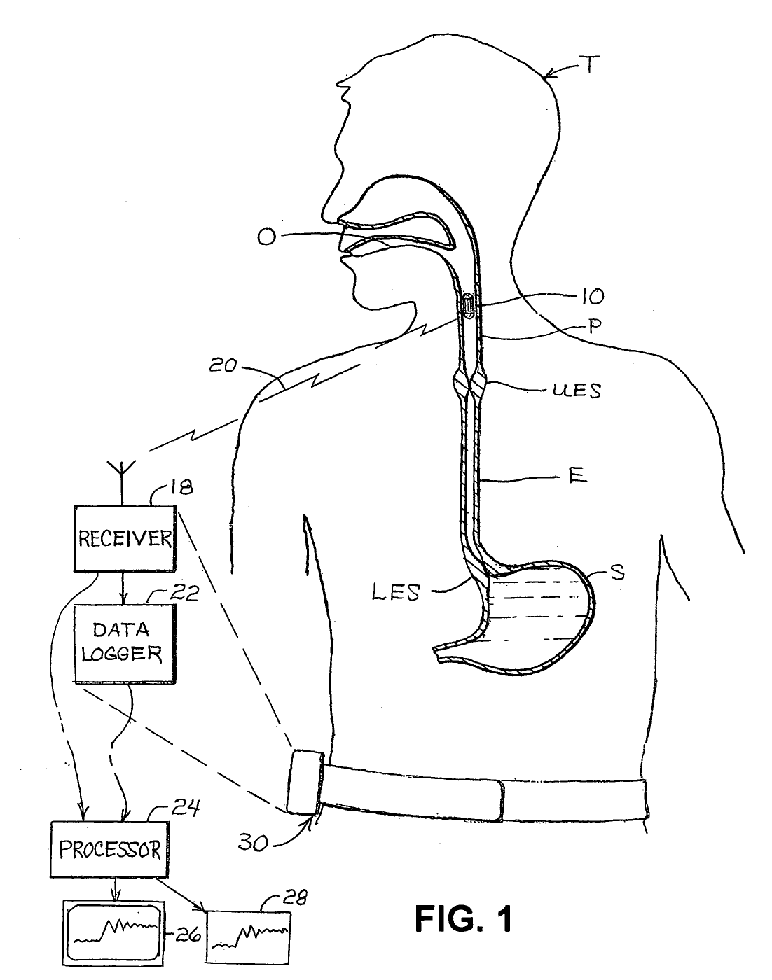

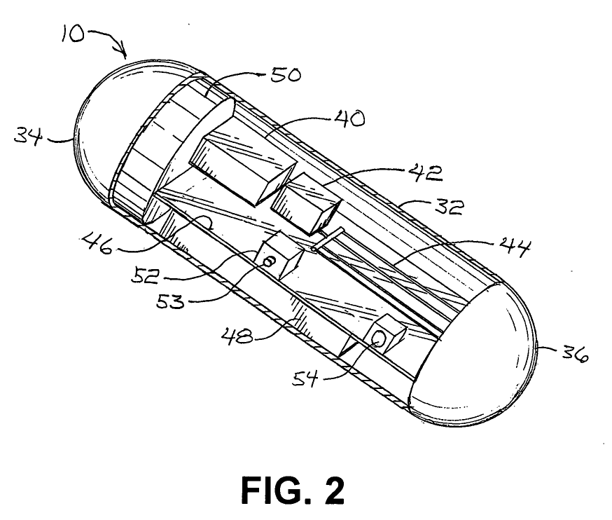

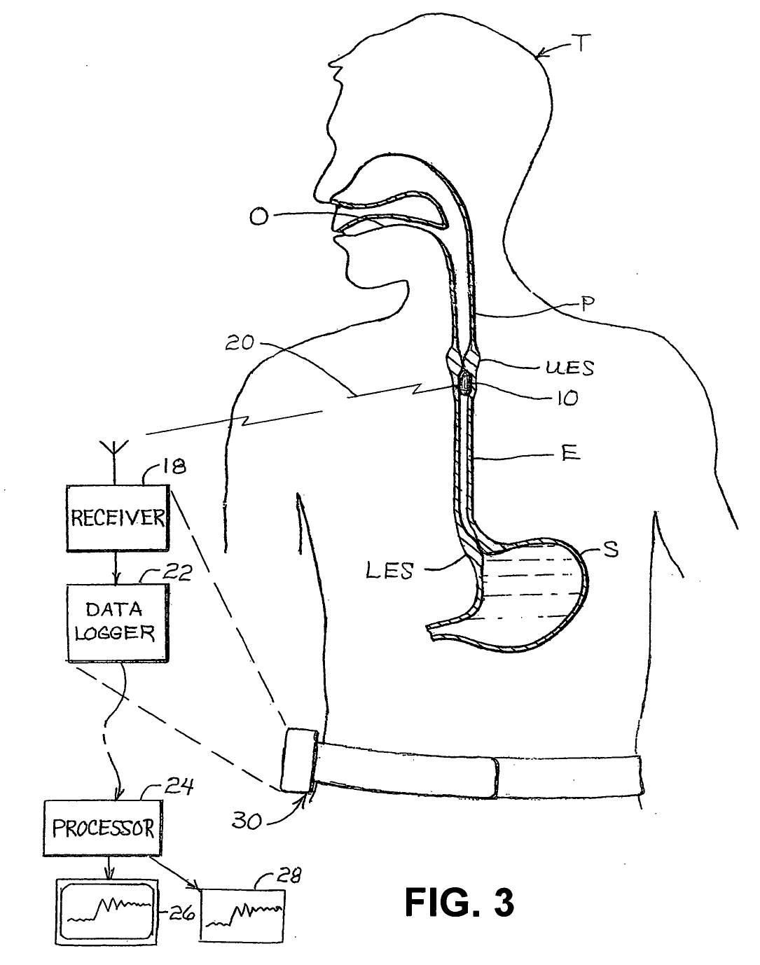

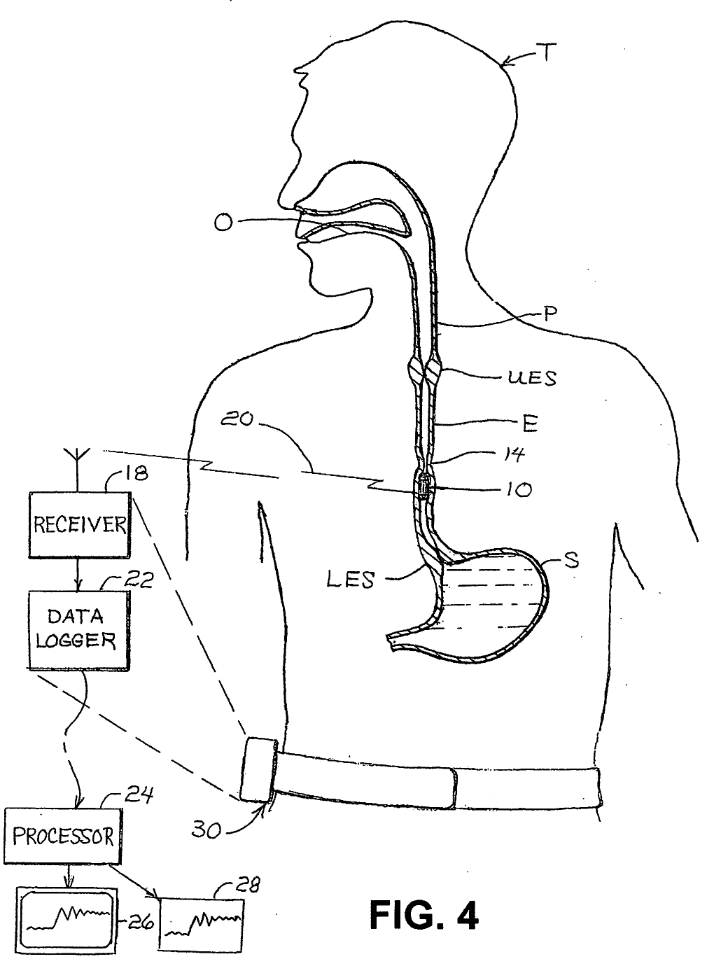

An electronic capsule 10 being swallowed to become a bolus in the gastrointestinal tract of a human test subject T is shown in FIG. 1 as it passes from the test subject's oral cavity O into the pharynx P on its way toward the stomach S. The capsule 10, illustrated diagrammatically in FIG. 2, contains inertial navigation sensors (accelerometers or gyroscopes) 40 for sensing magnitude of acceleration, a RF (radio frequency) signal transmitter 42 and antenna 44 for transmitting acceleration data from the accelerometer 40 to a RF receiver 18 that is preferably positioned outside the body of the test subject T. Preferably, three accelerometers are used to determine acceleration in three dimensions, and also gyroscopes may be used to determine orientation. The receiver 18 receives RF signals 20 that are transmitted from the capsule, conditions, amplifies, and processes the signals, and sends them to a data logger 22, which records and stores the acceleration data for either real time or subsequent processing, calculations, display, or whatever other analysis techniques are desired. Therefore, as the capsule 10 progresses as a bolus through the gastrointestinal tract, as illustrated for example in several sequential positions in the esophagus E shown in FIGS. 3 and 4, its acceleration and orientation can be monitored, recorded, and observed in real time, if desired, or at a later time. A computer processor 24, visual display device 26, and printer/plotter device 28 can be used for data processing, calculations, and display functions including calculating and displaying velocity and positions of the capsule 10 bolus as well as accelerating forces acting on it as it moves through the gastrointestinal tract or through any portion of the gastrointestinal tract. The additional velocity, position, and force parameters can also be calculated and viewed in real time, if desired, or at a later time. The receiver 18 and data logger 22 can be positioned anywhere in proximity to the test subject T, or, if desired, can be attached to the body of the test subject T, such as with a belt assembly 30, as depicted in FIG. 1.

The capsule 10 is illustrated in FIG. 2 with a portion of its sidewall 32 shown cut away to reveal the principal components inside. The capsule 10 preferably has a slightly elongated, cylindrical shaped side wall 32 with rounded, hemispherical ends 34, 36, so that it is smooth and easy to swallow and tends to be oriented longitudinally by the tubular pharynx P and esophagus E, as shown in FIGS. 1, 3, and 4. Inertial navigation sensors 40 are positioned in the capsule 10 for sensing and measuring the acceleration of the capsule 10 in three dimensions as it is propelled through the gastrointestinal tract. The measured acceleration can be positive values indicating capsule 10 bolus is speeding up, negative values indicating it is slowing down, on zero indicating it is either moving at a constant speed or at a standstill. While there are many kinds of small or MEMS (micro-electro-mechanical systems) accelerometers and other small accelerometers that would work for this invention, such as piezo film, electromechanical servo, piezoelectric, liquid tilt, bulk micromachined piezoresistive, capacitive, micromachined capacitive, and others that are well-known and readily available, the example of this invention described herein includes MEMS capacitive accelerometers 40, such as the ADXL202E capacitive MEMS accelerometer available from Analog Devices, Inc., Norwood, Mass., which is believed to have some of the better attributes and characteristics for this application as explained in Appendix A. However, for purposes of this invention, the term “accelerometer” is meant to include any apparatus or device capable of measuring the acceleration of the capsule, regardless of how it is made, packaged, or used.

As mentioned above, the inertial navigation sensors 40 sense and measure the acceleration of the capsule 10 as it is propelled as a bolus through the gastrointestinal tract or through parts thereof. An accelerometer is an example of an inertial navigation measurement. In the pharyngeal phase of a typical swallow, as depicted in FIG. 1, the force of the tongue on the capsule 10 causes the first significant acceleration of the capsule 10. The second significant acceleration of the capsule 10 occurs in the UES region, as depicted, for example, in FIG. 3. The acceleration typically decreases as peristaltic contractions 14 of the esophagus E along with gravity push the capsule 10 through the more distal portions of the esophagus E approaching the stomachs, as depicted in FIG. 4, possibly due to the capsule 10 bolus encountering increasing abdominal pressure on the walls of the esophagus E in the more distal portions of the esophagus E. Such typical acceleration measurements with the capsule 10 during swallows are described in more detail in the attached Appendix A. Significant variations of such acceleration profiles and/or in velocity, position, or force parameters derived from the acceleration measurements in a particular test subject as compared to normal data acquired from large numbers of test subjects could indicate possible maladies, dysfunctions, or diseases.

Many of the commercially available accelerometers that can be used in this invention are made and sold in a package that includes an acceleration sensor element of a type mentioned above as well as an electronic circuit or chip closely coupled to the sense element to accurately measure the miniature changes in capacitance (or resistance, voltage, or amperage, depending on the type of accelerometer used) caused by the changes in acceleration and to convert such changes into useful electric signals. Therefore, it is not necessary to describe the precise electric circuits needed to provide such signals from the accelerometer, since they are readily available commercially. The inertial navigation sensor 40 will output useful signals that are indicative of the acceleration on a real time basis. Such signals can be digital or analog, depending on the manufacturer or characteristics and capabilities of the particular inertial navigation sensor used. For example, but not for limitation, a digital signal may comprise a pulse stream, the frequency or pulse density of which is proportional to the acceleration, while an analog signal may comprise a differential voltage output that is proportional to acceleration. An example inertial navigation sensor 40 is described in more detail in Appendix A below.

The inertial navigation sensor output signal could be collected and stored in the capsule 10 for later read-out and analysis after recovery of the capsule 10 from the test subject's gastrointestinal tract, but it is usually more desirable to monitor the acceleration signals on a real time basis as the capsule 10 moves through the gastrointestinal tract. Therefore, as mentioned above, a RF transmitter 42 can be provided in the capsule 10 and connected to the inertial navigation sensors 40 for transmitting the signals from the accelerometer 40 in a wireless manner to a receiver 18 located outside of the test subject's body. A loop antenna 44 comprising a copper, aluminum, or other electrically conductive trace on a printed circuit board 46 or any other suitable antenna structure can be connected to the transmitter 42 to transmit the signals 20 to the receiver 18. An example transmitter for data transmission is described in Appendix A, although there are numerous other available transmitters that would also work for this invention. A crystal 48 for reference phase-locked-loop frequency is included in the capsule 10 for use with the example transmitter 42.

While it is preferred to transmit the inertial navigation sensor measurement data on a real time basis, it could be stored for short periods of time and then transmitted in bursts or packets of accumulated acceleration measurement data. For example, the acceleration data measured during the capsule 10 transit through the pharynx and esophagus could be retained in a memory chip (not shown) in the capsule 10 and then transmitted in a burst or packet of data after the capsule enters the stomach S. Persons skilled in the art can easily implement these variations.

An example RF receiver 18 is also described in Appendix A, although many other available receivers would also be satisfactory. Likewise, persons skilled in the art are well aware of suitable data loggers 22, computer or microprocessors 24, display devices 26, printers/plotters 28, and how to use them to record, process, and display the acceleration data as well as the velocity, position, and force data that is derivable from the acceleration data. Also, as indicated by the alternate connection from the receiver 18 to the processor 24 in FIG. 1, the receiver acceleration measurement data can be fed directly from the receiver 18 to the processor 24 for data processing, calculations, and display.

As also explained in Appendix A, it is desirable to seal the capsule 10 during manufacture to prevent moisture intrusion and resulting current leakage from the battery 50, so a suitable switch or actuation device 52 to turn on the electric components in the capsule 10 without having to open the capsule 10 is desirable. While an example of a magnetic reed switch is described for that function in Appendix A, there are many other suitable switch mechanisms that could be used. For example, but not for limitation, the switch 52 could be a microswitch with an actuation button 53 in contact with or in proximity to an elastically deformable cylindrical wall 32 (shown partially cut away in FIG. 2 to reveal internal components). Therefore, pressing on the wall 32 adjacent the microswitch 52 with enough force to deform the wall 32 and thereby depress the button 53 could actuate the switch 52 to turn on the electric circuits and components in the capsule 10. Again, there are so many ways of turning on the electric circuit that this invention is not limited to any particular switch method or apparatus. An LED (light emitting diode) 54 positioned adjacent a transparent window portion (not shown) in the wall 32 can be used to show that the circuits are turned on and powered by the battery 50.

Example methods for calculating other parameters of velocity, position, and force from the acceleration data are also explained in Appendix A. Any processor 24 can be used to calculate any one or more of these parameters for any instant during the time in which the acceleration measurements are made and over periods or increments of such time. These calculated parameters can also be displayed in real time or later in still frames.

Since numerous modifications and combinations of the methods and embodiments described above and in the attached Appendix A will readily occur to persons skilled in the art, once they become familiar with and understand the principles of this invention, the invention is not limited to the specific structures and methods described herein and in Appendix A. Also, while the illustrations and examples described herein and in Appendix A are shown in relation to the oral cavity, pharynx, and esophagus above the stomach, this invention is also applicable to the rest of the gastrointestinal tract, and it works for retrograde as well as antegrade bolus movement.

Appendix A

The example of an ESO-Pill described in this appendix is a MEMS device comprising of miniature inrtyisl navigation sensors such as accelerometers and associated electronic conditioning circuit 40; RF transmission device 42; and battery power supply 50. These components are positioned within a swallowable capsule 10. The RF data transmitted in real time by the pill are received by a receiver 18 connected to a standard wireless data logger 22 attached to the patient which is battery supplied. The patient is given one or several ESO-Pills along with a protocol of how to take them. The pill is stored in a magnet housing and turns itself on upon the removal from the housing. The pill has a lifetime of about half hour, significantly longer than the time needed to travel through the esophagus. This relatively short lifetime reduces battery requirements, a rather important benefit considering the limited volume of the device. Since the acceleration in the esophagus is the only acceleration level in the gut that can be assessed with the presently available micro-accelerometers, the Eso-Pill would “die” upon entering the stomach, which would be indicated by a final abrupt change in the acceleration.

MEMS accelerometers have been used in a wide variety of applications. With advanced MEMS technologies, accelerometers and signal conditioning circuitry 40 are monolithically integrated into a single chip resulting in improved performance, miniaturization, and reduced cost compared to conventional force balance accelerometers. Based on measuring principles, MEMS accelerometers can be segregated into two different types: piezoresistive and capacitive. In piezoresistive-based accelerometers, silicon resistors, which change electrical resistance in response to applied mechanical load, are connected in a Wheatstone bridge to produce a voltage proportional to the acceleration of the proof mass attached to the sensor housing. In contrast, in capacitive accelerometers acceleration is measured by the change in capacitance due to a moving plate attached to the proof mass. Capacitive accelerometers are more popular than piezoresistive designs due to several advantages they have, including high sensitivity, high resolution, low noise, low drift, stable DC characteristics, low power dissipation, and low temperature drift. For esophageal applications, a resolution of at least 6.88 cm/s² (10 % of the minimal acceleration value in the esophagus, see Table 1) is essential, i.e. the accelerometer has to detect acceleration levels as low as 6.88 cm/s². This, along with the obvious power supply and dimension limits narrows the technological availability to our application. Hence, a miniature-sized capacitive MEMS accelerometer, featuring low power consumption and high resolution, is a suitable candidate for the pill 10, as for example the ADXL202E capacitive MEMS accelerometer from Analog Devices (Norwood, Mass.), which features also a low cost, an advantage that might become important if more than one pill has to be swallowed by the patient over a certain time period in order to provide averaged estimates of the bolus transit in the esophagus. The size of the accelerometer is 5 mm×5 mm×2 mm, with a resolution of 2 mg (1 g=981 cm/s²) at 60 Hz, wide range power supply from 3 V to 5.25 V, and current consumption of less than 1 mA. Taking into account that the longevity of the ESO-Pill is in the range of half hour, these characteristics were considered completely satisfactory.

TABLE 1
Bolus motility in esophagus

| | | Mean Acceleration |
|---|---|---|
| Mean Transit Time (s) | Mean Velocity (cm/s) | (cm/s²) |
| 4.2 ± 0.9 | 9.6 ± 1.4 | 84.2 ± 15.4 |

To remotely monitor the bolus transit in real-time in the esophagus, a reliable and effective wireless radio frequency (RF) link has to be established. Since the size of the transmitter is important due to the limited space in the pill, a monolithically integrated transmitter chip is favored. In the example given in this appendix, the MAX1472 ASK transmitter from Maxim (Sunnyvale, Calif.) is chosen. The MAX1472 is a crystal-referenced phase-locked-loop (PLL) transmitter which operates in the 300 MHz to 450 MHz frequency range (VHF/UHF). The transmitter is available in a 3 mm×3 mm SOT23 package and is capable of delivering +10 dBm output power with a current consumption of less than 9.1 mA. A small-loop antenna fabricated out of a copper trace on the PC board can be employed to emit the modulated signal, and with a careful design of the matching network, maximum performance of the transmitter may be achieved. In addition, the transmitter can be directly coupled to the output of the ADXL202E accelerometer, which reduces the cost and saves additional space by avoiding the conditioning circuitry. An ASK superheterodyne receiver such as MAX1470 or MAX1473 is used to receive the transmitted data and send it to a data logger.

The functionality and lifetime of the ESO-Pill are limited by the capacity of the power supply. Since the amount of energy that a battery can deliver is proportional to its size, a precise analysis of the power consumption of the entire system needs to be performed in order to achieve a compromise between the capacity and the size of the battery. Table 2 lists the power consumption of the accelerometer and the RF transmitter described in this appendix. In the present analysis, the worst-case scenario is used, e.g. it is assumed that input data to the transmitter is always in logic high, devices are operating at the highest tolerable temperature, etc. Note that in contrast to the accelerometer and the RF transmitter, power dissipation in other passive components is negligible. Table 2 shows that the system consumes 52.2 mW power at a voltage level of 3 V. As a result, a 9.5 mm×2.7 mm 3 V lithium (MnO2/Li) cell battery delivering energy rated at 90 mWh is selected to serve as the power supply source of the entire system.

Since the pill should be firmly sealed to prevent current leakage or damage to the device, activation in a wireless manner is preferred. A miniature-sized reed switch may be used as the turn-on mechanism. The reed switch is configured to be normally closed, and in order to maintain the switch open, the pill needs to be stored in a permanent magnetic housing. Upon removal of the pill from the housing, the switch is closed and the system is activated. In the specific design of this appendix, the HSR-502 reed switch from Hermetic Switch Inc. (Chickasha, Okla.) is preferred due to its form C contact arrangement (the switch can be configured to be either normally closed or normally open) and miniaturized size. The fact that the pill is turned on is confirmed by a light-emitting diode (LED). Thus, the energy consumption of the overall system can be re-estimated at 172.2 mW, which still can be easily supported by the chosen lithium cell battery.

TABLE 2
Analysis of the power consumption

| | Power Consumption |
|---|---|
| | (using a 3 V battery) |
| Active Components | [mW] |
| ADXL202E Accelerometer | 3.0 |
| MAX1472 RF Transmitter | 49.2 |
| Entire System | 52.2 |

The acceleration may be the only parameter delivered by the ESO-Pill wirelessly to the external data logger. Therefore, it is important to clearly understand what to expect from it, so that successful integrations for determining the velocity of propagation and the displacement of the pill are possible.

Swallowing is a complex physiological process that is responsible for transporting the food from the mouth to the stomach and involves three phases: 1) the oral phase, 2) the pharyngeal phase, and 3) the esophageal phase. In the oral phase, the process of swallowing starts in voluntarily mixing and chewing of food in the oral cavity. The pharynx has two parts: 1) the oropharynx and 2) the hypopharynx, divided by the superior edge of the epiglottis. The movement of the bolus into the oropharynx driven by the tongue base force and gravity (as the subject is seated in the upright position) is defined as the start of the pharyngeal phase. As the bolus leaves the pharyngo-esophageal (PE) region (also known as the UES) and enters the esophagus, the pharyngeal phase ends and the esophageal phase starts. The departure of the bolus from the LES indicates the end of the esophageal phase.

In 1990, Ku et al. showed that the acceleration of the bolus traversing the pharynx, the UES, and the esophagus was variable, and this variability was found to be location-dependent. In the pharyngeal phase of swallowing, the first peak of the acceleration occurs while the bolus traverses the base of the tongue to the level of the epiglottis due to the superior tongue base force and the gravity. The acceleration quickly decreases to below zero while the bolus traverses the area located at the laryngeal introitus. After the bolus passes this area, its acceleration significantly increases again to a second peak in the UES region. Following the entry of the bolus into the esophagus, the acceleration slows down and reaches the lowest level in the distal third of the esophagus due to increased abdominal pressure. The first peak of the acceleration has a magnitude of 400-500 cm/s² based on measurements with the use of a 10 cm³ liquid bolus head and the subject seating in an upright position, and the second peak has a magnitude of 300-400 cm/s². In fact, when the ESO-Pill is swallowed, it acts as a 1.727 cm³ solid bolus, and it was concluded by Kern et al. that the kinematics of a solid bolus are similar to that of a liquid bolus head. Therefore, a similar behavior to the liquid bolus head is expected from the pill.

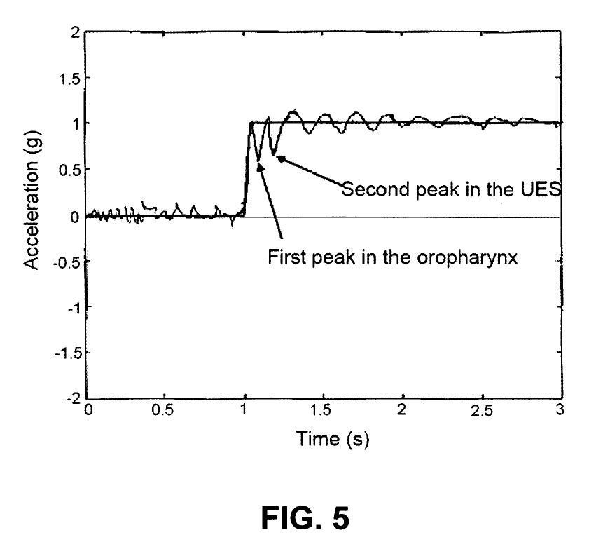

FIG. 5 shows the anticipated plot that describes the bolus acceleration in a swallowing process. In the beginning, while the pill is in a horizontal position in the mouth of a subject, the measured acceleration is approximately zero with small fluctuations due to random movements. As the pill enters the oropharynx, it quickly turns itself to a vertical position and the first peak occurs at about the same time, and the offset is also shifted from zero to 1 g due to gravity. The time right before when the first peak occurs can be defined as the initial starting point. The second peak shown in FIG. 5 corresponds to the acceleration of the pill in the UES region.

The acceleration curve can be used to calculate the bolus velocity and displacement by applying discrete integration. The velocity can be calculated using the central difference scheme:
vᵢ₊₁=2(Δt)aᵢ+vᵢ₋₁ cm/s, (1)
where aᵢ is the acceleration (cm/s²) at the ith instant, Δt is the length of time between two successive time instants and its value depends on the sampling frequency, and vᵢ₊₁ and vᵢ₋₁ are the velocities at the (i+1)ₜₕ and (i−1)ₜₕ instants, respectively. Similarly, the displacement can be obtained from the velocity values as
dᵢ₊₁=2(Δt)vᵢ+dᵢ₋₁ cm. (2)

In addition, since the mass and the acceleration of the pill are both known, the force exerting on the pill at each time instant can be determined by employing Newton's Second Law, force equals mass times acceleration.

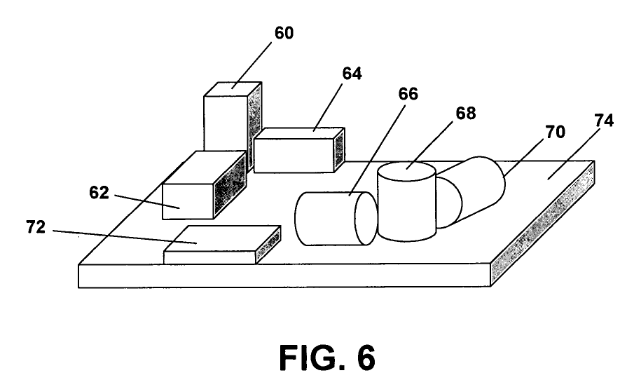

An exemplary inertial navigation sensor 40 is shown in FIG. 6, which includes three mutually orthogonal accelerometers 60, 62 and 64 forming an acceleration measurement unit, three mutually orthogonal gyroscopes 66, 68 and 70 forming an attitude measurement unit, and a signal conditioning unit 72, all supported on a base 74. In a minimalistic setup, the sensor 40 can contain a single accelerometer oriented along the direction of motion. In a maximalistic setup, it would contain three mutually orthogonal accelerometers and three mutually orthogonal attitude sensors. Data from the sensor 40 is sent to a computer as described above, which computes the navigation parameters based on the measurements of acceleration and attitude sensors. The operation involves the measurement of force usually by an orthogonal triad of accelerometers. The orientation of this accelerometer triad relative to a predefined coordinate frame is established and maintained by three (usually) orthogonal gyroscope axes. An accelerometer reacts to the actual acceleration and gravity without having the means to distinguish between them. Therefore, its output is not the actual acceleration but rather is known as the “specific force”, \overline{f}. The specific force is the difference between the real acceleration of the capsule 10 and the gravity which acts on the accelerometer as well.

In order to be able to calculate the specific force, knowledge of the gravitational vector, \overline{g}ₘ, is necessary. Since the orientation of the accelerometer relative to the Earth is assumed to be known, the gravity component can be removed. Based on Newton's second law, the basic inertial equation is:
\overline{\ddot{r}}|ᵢ=\overline{f}+\overline{g}ₘ
where \overline{\ddot{r}}|ᵢ is the real actual acceleration of the carrying vehicle. This acceleration vector is obtained by the second time derivative of the position vector \overline{r} in the inertial (i) coordinate frame.

The attitude sub-system provides the temporary angular status of the accelerometer-sensitive axis with relation to an external predefined reference frame. Knowledge of the actual angular status when the acceleration is measured is required to perform proper integration and obtain accurate velocity and position of the INS with respect to the required reference frame. The angular status is changing as a function of the angular velocity:
C_{b}ⁱ=C_{b}ⁱ·Ω_{ib}^{b}
where:
C_{b}ⁱ—is the Directional Cosine Matrix (DCM) which transforms between the body axis reference frame, to the inertial reference frame, i.
Ω_{ib}ⁱ—is the skew symmetric matrix of the angular velocity vector (≡w_{ib}^{b}×, where the × stands for a cross product). The vector W_{ib}^{b} describes the angular velocity of the body coordinate frame with reference to the inertial coordinate frame as observed in the body frame.

In the example shown, where the sensors are attached and affixed to the body 74, the angular reference frame is obtained using the output of the gyros, W_{ib}^{b}. Applying these measurements to the equation in paragraph 35 provides the required angular transformation. The computer calculates the temporary attitude from the measured angular velocity according to the readings from the gyros. This attitude information is used to transform the accelerometer outputs from the body axis (where they are measured) to the required axis of the reference frame. The navigation computer solves the basic acceleration equation by calculating the gravitation vector \overline{g}ₘ, using a given model, and adding this value to the measured specific force vector \overline{f}. Based on the gyro measurements, the equation in paragraph 35 derives the proper attitude relations. With these attitude relations, the navigation computer can implement the proper spatial projections of the acceleration measurements, and by integrating of these projection components, can obtain the velocity (either the inertial velocity \overline{u}, or the velocity relative to the Earth coordinate frame \overline{v}).
\overline{u}=\dot{\overline{r}}|ᵢ
\overline{v}=\dot{\overline{r}}|ₑ

For position navigation, the kinematic system presentation includes a second-order vector differential equation of the position \overline{r} and an algebraic equation where \overline{r} and its derivatives are used to obtain the inertial velocity \overline{u}, or the velocity relative to Earth \overline{v}:
\overline{\ddot{r}}|ᵢ=\overline{f}+\overline{g}ₘ
\overline{u}=\overline{\dot{r}}|ᵢ

The calculation is computed in a predefined coordinate frame q, which has a certain angular velocity relative to the inertial coordinate frame. According to the principles of differentiating vectors in frames which are in relative rotation (Coriolis Theorem), the relation between those quantities, acceleration and velocity, is:
\overline{\dot{r}}|ᵢ=\overline{\dot{r}}|_{q}+\overline{w}_{iq}×\overline{r}
\overline{\ddot{r}}|ᵢ=\overline{\ddot{r}}|_{q}+2·\overline{w}_{iq}×\overline{\dot{r}}|_{q}+\overline{\dot{w}}_{iq}|_{q}×\overline{r}+\overline{w}_{iq}×(\overline{w}_{iq}×\overline{r})
where \overline{w}_{iq} represents the vector of the relative angular velocity of the q-coordinate frame with reference to the inertial coordinate frame as observed in the q-coordinate frame.

Using the previous relations it can be found that:
\overline{v}=\overline{\dot{r}}|_{q}+\overline{w}_{eq}×\overline{r}
\overline{\ddot{r}}|_{q}+2·\overline{w}_{iq}×\overline{\dot{r}}|_{q}+\overline{\dot{w}}_{iq}|_{q}×\overline{r}+\overline{w}_{iq}×(\overline{w}_{iq}×\overline{r})−\overline{w}ᵢₑ×({overscore (w+EEᵢₑ×\overline{r})=\overline{f}+\overline{g}

For velocity navigation, the system kinematics is described by a pair of first-order differential equations, one for the position and the other for the velocity. Following the basic inertial equation:
\overline{\dot{r}}|ᵢ=\overline{u}
\overline{\dot{u}}|ᵢ=\overline{f}+\overline{g}ₘ
Using the rules for differentiating vectors in rotating frames results in:
\overline{\dot{u}}|ᵢ=\overline{\dot{u}}|_{q}+\overline{w}_{iq}×\overline{u}
When applying the relation between the inertial velocity and the velocity relative to the Earth:
\overline{u}=\overline{v}+\overline{w}ᵢₑ×\overline{r}
the navigation equations pair can be derived:
\overline{\dot{v}}|_{q}=−(\overline{w}_{iq}+\overline{w}ᵢₑ)×\overline{v}+\overline{f}+\overline{g}
\overline{\dot{r}}|_{q}=\overline{v}−\overline{w}_{eq}×\overline{r}

The description of paragraphs 33-40 is a summary of known art in the art of inertial navigation systems and may be used in conjunction with data from the capsule 10 by the computer 24 to compute the position, velocity, acceleration and orientation of the capsule 10.

Immaterial modifications may be made to the specific example described here without departing from the invention.

What is claimed is:

1: Apparatus for monitoring transit of a bolus in a gastrointestinal tract of a test subject's body, comprising:
a capsule sized and shaped for being swallowable and movable through the gastrointestinal tract, said capsule containing a set of inertial navigation sensors that are capable of sensing, measuring or estimating acceleration, velocity and postion of the capsule on a real time basis as the capsule moves through the gastrointestinal tract or through portions of the gastrointestinal tract.

2: The apparatus of claim 1, including a RF transmitter in the capsule connected to receive electric signals indicative of inertial navigation measurements from the set of inertial navigation sensors and to transmit RF signals that are indicative of the acceleration, velocity and position measurements in a wireless manner.

3: The apparatus of claim 2, including a RF receiver positioned outside the test subject's body and configured to receive the RF signals transmitted by the transmitter and to convert the RF signals to electric signals.

4: The apparatus of claim 3, including a data logger connected to the receiver for collecting and recording the inertial navigation measurement data.

5: The apparatus of claim 4 including a processor programmed to receive acceleration data, time, and distance data and to calculate one or more of the following: velocity of the capsule, position of the capsule, or net force on the capsule that causes the movement of the capsule.

6: The apparatus of claim 1, wherein the capsule is sealed to prevent leakage of fluids in the interior of the capsule.

7: The apparatus of claim 6, including switch means inside the capsule for actuating the set of inertial navigation sensors by an action outside the capsule without breaking the capsule seal.

8. The apparatus of claim 1, wherein the capsule is elongated, so that it gets oriented to longitudinal alignment with the gastrointestinal tract, and the inertial navigation sensors are oriented in such way that a complete navigation solution for the moving or stationary capsule is always available.

9: Apparatus for monitoring transit of a bolus in a gastrointestinal tract of a test subject, comprising a capsule that is sized and shaped for swallowing and transit through the gastrointestinal tract, characterized by:
Inertial navigation means in the capsule for measuring and/or estimating acceleration, velocity and position of the capsule on a real time basis as the capsule moves through at least a portion of the gastrointestinal tract.

10: The apparatus of claim 9, including transmitter means in the capsule for transmitting signals indicative of the measurements or the estimates produced by the set of inertial navigation sensors.

11: The apparatus of claim 10, including receiver means positioned outside the test subject's body for receiving the signals transmitted by the transmitter.

12: The apparatus of claim 11, including processor means for calculating one or more of the following parameters: acceleration of the capsule, velocity of the capsule; position of the capsule; or net force on the capsule that causes the motion of said capsule.

13: A method of monitoring bolus movement through at least a portion of a gastrointestinal tract of a test subject, comprising:
the test subject swallowing a set of inertial navigation sensors that measures and/or estimated acceleration, velocity and position on a real time basis and produces electric signals that are indicative of these measurements; and
collecting the said measurements for analysis and display.

14: The method of claim 13, including transmitting the inertial navigation measurements from inside the gastrointestinal tract to a receiver located outside the gastrointestinal tract.

15: The method of claim 14, including transmitting the inertial navigation measurements in real time.

16: The method of claim 14, including accumulating the inertial navigation measurements over a period of time and then transmitting in bursts or packets of inertial navigation measurements.

17: The method of claim 14, including receiving the transmissions of inertial navigation measurements outside the test subject's body.

18: The method of claim 17, including recording the inertial navigation measurements in a data logger.

19: The method of claim 18, including processing the inertial navigation measurements with algorithms to derive one or more of the following parameters: acceleration of the capsule moving in the gastrointestinal tract, velocity of the capsule moving in the gastrointestinal tract; position of the capsule in the gastrointestinal tract; and net force that moves the capsule in the gastrointestinal tract.

20. The method of claim 13 including enclosing the set of inertial navigation sensors in a capsule that is sized and shaped for swallowing and moving through the gastrointestinal tract.
